SEQUENCE LISTING (74 sequences)

SEQ ID NO: 1 - DNA, 23 bp, Artificial Sequence
  Acceptor A1(23 nt)
gatgtgtccgtgcnnnggttcga

SEQ ID NO: 2 - DNA, 100 bp, Artificial Sequence
  LibraryL1(100nt)
ggaagagatggcgacnnnnnnnnnnnnnnnnnnnnnnnnnnnnnnnnnnnnnnnnnnnnnnnnnnnnnnnnnnnnnnnnnnnnnnagctgatcctgatgg

SEQ ID NO: 3 - DNA, 38 bp, Artificial Sequence
  Template T1
gtcgccatctcttcctcgaaccatagcacggacacatc

SEQ ID NO: 4 - DNA, 38 bp, Artificial Sequence
  Primer 1 (P1)
gaattctaatacgactcactataggaagagatggcgac

SEQ ID NO: 5 - DNA, 15 bp, Artificial Sequence
  Primer 2 (P2)
ccatcaggatcagct

SEQ ID NO: 6 - DNA, 23 bp, Artificial Sequence
  Primer 3 (P3), ribo-terminated
gaattctaatacgactcactatn

SEQ ID NO: 7 - DNA, 122 bp, Artificial Sequence
  pH3DZ1
gatgtgtccgtgcnnnggttcgaggaagagatggcgacagttggcgaagatcggtagtacgaggaaatagggggtgagtggtgtaggcttgaaggtgccacgtcgagagctgatcctgatgg

SEQ ID NO: 8 - DNA, 121 bp, Artificial Sequence
  pH3DZ2
gatgtgtccgtgcnnnggttcgaggaagagatggcgactggtataagggaggctagagagggtgtggaagagcggacaaagggtggattgttaggtatattatttgagctgatcctgatgg

SEQ ID NO: 9 - DNA, 123 bp, Artificial Sequence
  pH3DZ3
gatgtgtccgtgcnnnggttcgaggaagagatggcgacgaggtgaacaagcggctgagcttttggaagaaggcataaggaaaaggttagataaaggtgctggtgcgatagctgatcctgatgg

SEQ ID NO: 10 - DNA, 121 bp, Artificial Sequence
  pH3DZ4
gatgtgtccgtgcnnnggttcgaggaagagatggcgacttgggaggaggagactgatatttggtcctttttagccggtgccgttttaggttgtggggtgggtggtaagctgatcctgatgg

SEQ ID NO: 11 - DNA, 123 bp, Artificial Sequence
  pH3DZ5
gatgtgtccgtgcnnnggttcgaggaagagatggcgactggtggaggaaagaaatagcttcgtcttccatcgtgatgagtggggagggaaaatgagtaggggtctgtaagctgatcctgatgg

SEQ ID NO: 12 - DNA, 121 bp, Artificial Sequence
  pH3DZ6
gatgtgtccgtgcnnnggttcgaggaagagatggcgacctagagtagctttgtgctgtaaggctagttttggtaaagatagggctctatggtaccggtttggctatagctgatcctgatgg

SEQ ID NO: 13 - DNA, 121 bp, Artificial Sequence
  pH3DZ7
gatgtgtccgtgcnnnggttcgaggaagagatggcgacgtggtgtgtgatagggagccaacgagtgacgagataggtagccacggttaggattggaaggattgtacagctgatcctgatgg

SEQ ID NO: 14 - DNA, 122 bp, Artificial Sequence
  pH3DZ8
gatgtgtccgtgcnnnggttgaggaagagatggcgacggcaaaaggaaacgcagtttgggtggaaacaggtggaagggtgtcacgagttagtggagtcgaccccgtgagctgatcctgatgg

SEQ ID NO: 15 - DNA, 123 bp, Artificial Sequence
  pH3DZ9
gatgtgtccgtgcnnnggttcgaggaagagatggcgacggaagttggagggttcgtattgctacgttgccttagagaggttgtggaagagcggcacatcattgttgggagctgatcctgatgg

SEQ ID NO: 16 - DNA, 123 bp, Artificial Sequence
  pH3DZ10
gatgtgtccgtgcnnnggttcgaggaagagatggcgactgaatgaaacctcgggcataaattacggaaacggctttaattttttagtggaaagatccgataacgaggtagctgatcctgatgg

SEQ ID NO: 17 - DNA, 121 bp, Artificial Sequence
  pH3DZ11
gatgtgtccgtgcnnnggttcgaggaagagatggcgacgaagtgggggtcgggggaagggaggcacgcgtaaaggtaggtgtgagggcgggtgagagttggacaatagctgatcctgatgg

SEQ ID NO: 18 - DNA, 123 bp, Artificial Sequence
  pH4DZ1
gatgtgtccgtgcnnnggttcgaggaagagatggcgactgtatgctagctcggggagaaacatctttgcgggataaggccgccgatagagcggaagcgacttggttgtagctgatcctgatgg

SEQ ID NO: 19 - DNA, 123 bp, Artificial Sequence
  pH4DZ2
gatgtgtccgtgcnnnggttcgaggaagagatggcgactgaatagggtcctaggcataaattacgaaaacggctttaatcttttagtggaaaggtccgataacgagtgagctgatcctgatgg

SEQ ID NO: 20 - DNA, 123 bp, Artificial Sequence
  pH4DZ3
gatgtgtccgtgcnnnggttcgaggaagagatggcgactgggtaaaaggaaaagatggcggagcgagttgatggcgtgattaggaggaggacttaaaggtggtggttgagctgatcctgatgg

SEQ ID NO: 21 - DNA, 121 bp, Artificial Sequence
  pH4DZ4
gatgtgtccgtgcnnnggttcgaggaagagatggcgactgtacggtagggagggtgcaaggtgaatcggattagtttacggaagagtgtgattgagtccgatagctagctgatcctgatgg

SEQ ID NO: 22 - DNA, 122 bp, Artificial Sequence
  pH4DZ5
gatgtgtccgtgcnnnggttcgaggaagagatggcgaccagtagggcaatttggttgggtttaatgtgatacgaagaaccatatttgcggagttctagccggccgatagctgatcctgatgg

SEQ ID NO: 23 - DNA, 122 bp, Artificial Sequence
  pH4DZ6
gatgtgtccgtgcnnnggttcgaggaagagatggcgacaagatggggctctgacgaggagtttagcggtgatccctgaggacgtttgttgatggatgtggttgggtaagctgatcctgatgg

SEQ ID NO: 24 - DNA, 123 bp, Artificial Sequence
  pH4DZ7
gatgtgtccgtgcnnnggttcgaggaagagatggcgacgaggtgggggtcgggggaagggaggcacgcgtaaaggtaggtgtgagggcgcatgagggaattggacgatagctgatcctgatgg

SEQ ID NO: 25 - DNA, 123 bp, Artificial Sequence
  pH4DZ8
gatgtgtccgtgcnnnggttcgaggaagagatggcgacgggagttggaggatccgtactgttacgttgtcttagagagggtgtggaagagcggcacattactgttgggagctgatcctgatgg

SEQ ID NO: 26 - DNA, 123 bp, Artificial Sequence
  pH5DZ1
gatgtgtccgtgcnnnggttcgaggaagagatggcgactgaatagggtctcgggcataaattacggaaacggttttaattttctagtggaaaggtccgataacgaggtagctgatcctgatgg

SEQ ID NO: 27 - DNA, 122 bp, Artificial Sequence
  pH5DZ2
gatgtgtccgtgcnnnggttcgaggaagagatggcgacctgtgggaggctaagagaggttgtggaagagcggtaaactaatatcagtgttatgacagtggtgattgcagctgatcctgatgg

SEQ ID NO: 28 - DNA, 122 bp, Artificial Sequence
  pH5DZ3
gatgtgtccgtgcnnnggttcgaggaagagatggcgaccggagtagggggggaaggttgggtaaaggaatttttatgctgttagcaggtctaacggcggtgcaggggagctgatcctgatgg

SEQ ID NO: 29 - DNA, 121 bp, Artificial Sequence
  pH5DZ4
gatgtgtccgtgcnnnggttcgaggaagagatggcgacggcagaggaggtgggcgatgagtagtagggggggaaggttgggttcagtttagttgcggttggtatacagctgatcctgatgg

SEQ ID NO: 30 - DNA, 123 bp, Artificial Sequence
  pH5DZ5
gatgtgtccgtgcnnnggttcgaggaagagatggcgacgaagtgggggtcgggggaagggaggcacgtgtaaaggtaggtgtgagggtgtatggaagagttggacaacagctgatcctgatgg

SEQ ID NO: 31 - DNA, 123 bp, Artificial Sequence
  pH5DZ6
gatgtgtccgtgcnnnggttcgaggaagagatggcgactcgtagggggggaaggttgggtggaaggagttagtaagacgattgtactagcggtgagggcagggtgatgagctgatcctgatgg

SEQ ID NO: 32 - DNA, 123 bp, Artificial Sequence
  pH6DZ1
gatgtgtccgtgcnnnggttcgaggaagagatggcgacatcggacaagggaggcactggaggttgaggtagtgagcgttggttaacgccggacaaagggaagcatggtagctgatcctgatgg

SEQ ID NO: 33 - DNA, 123 bp, Artificial Sequence
  pH6DZ2
gatgtgtccgtgcnnnggttcgaggaagagatggcgactcgtaaggggggaaggttgggtggagggagtcagtaagacgattgtactagtggtgagggcaggatgatgagctgatcctgatgg

SEQ ID NO: 34 - DNA, 122 bp, Artificial Sequence
  pH6DZ3
gatgtgtccgtgcnnnggttcgaggaagagatggcgacgagtgggggtcgggggaagggaggcatgcgtaaaggtaggtgcgagggtgcatgaaagggttgggcaacagctgatcctgatgg

SEQ ID NO: 35 - DNA, 122 bp, Artificial Sequence
  pH7DZ1
gatgtgtccgtgcnnnggttcgaggaagagatggcgacgtggaacccatgatgagccgagttggggtgtgtctctcgtatatggcggaagtgggacaatagttgagtagctgatcctgatgg

SEQ ID NO: 36 - DNA, 123 bp, Artificial Sequence
  pH7DZ2
gatgtgtccgtgcnnnggttcgaggaagagatggcgacgtcggacaagggaggcactggggattgaggtagtgagcgttggttaacgccggacaaaggggagcatggtagctgatcctgatgg

SEQ ID NO: 37 - DNA, 123 bp, Artificial Sequence
  pH7DZ3
gatgtgtccgtgcnnnggttcgaggaagagatggcgactaactcattatgaggtgacggagtaccggaagagggagagatgaagggatgggcggttgtgctgtgttggagctgatcctgatgg

SEQ ID NO: 38 - DNA, 122 bp, Artificial Sequence
  pH7DZ4
gatgtgtccgtgcnnnggttcgaggaagagatggcgaccccatgatgagagtgctactcggaagagggacatgataggggagggattagatggtgtttattgtgtacagctgatcctgatgg

SEQ ID NO: 39 - DNA, 76 bp, Artificial Sequence
  pH7DZ1S
gatgtgtccgtgcnnnggttcgaagaacccatgatgagccgagttttctcgtatatggcggaagtgggacaatagt

SEQ ID NO: 40 - DNA, 57 bp, Artificial Sequence
  E1
gaatcgaacccatgatgagccgagttttctcgtatatggcggaagtgggacaatagt

SEQ ID NO: 41 - DNA, 29 bp, Artificial Sequence
  E2A
gaatcgaacccatgatgagccgagttttt

SEQ ID NO: 42 - DNA, 34 bp, Artificial Sequence
  E2B
aaaaactcgtatatggcggaagtgggacaatagt

SEQ ID NO: 43 - DNA, 69 bp, Artificial Sequence
  pH3DZ1 - random
agttggcgaagatcggtagtacgaggaaatagggggtgagtggtgtaggcttgaaggtgccacgtcgag

SEQ ID NO: 44 - DNA, 69 bp, Artificial Sequence
  pH3DZ2 - random
tggtataagggagggctagagagggtgtggaagagcggacaaagggtggattgttaggtatattatttg

SEQ ID NO: 45 - DNA, 70 bp, Artificial Sequence
  pH3DZ3 - random
gaggtgaacaagcggctgagcttttggaagaaggcataaggaaaaggttagataaaggtgctggtgcgat

SEQ ID NO: 46 - DNA, 68 bp, Artificial Sequence
  pH3DZ4 - random
ttgggaggaggagactgatatttggtcctttttagccggtgccgttttaggttgtggggtgggtggta

SEQ ID NO: 47 - DNA, 70 bp, Artificial Sequence
  pH3DZ5 - random
tggtggaggaaagaaatagcttcgtcttccatcgtgatgagtggggagggaaaatgagtaggggtctgta

SEQ ID NO: 48 - DNA, 68 bp, Artificial Sequence
  pH3DZ6 - random
ctagagtagctttgtgctgtaaggctagttttggtaaagatagggctctatggtaccggtttggctat

SEQ ID NO: 49 - DNA, 68 bp, Artificial Sequence
  pH3DZ7 - random
gtggtgtgtgatagggagccaacgagtgacgagataggtagccacggttaggattggaaggattgtac

SEQ ID NO: 50 - DNA, 70 bp, Artificial Sequence
  pH3DZ8 - random
ggcaaaaggaaacgcagtttgggtggaaacaggtggaagggtgtcacgagttagtggagtcgaccccgtg

SEQ ID NO: 51 - DNA, 70 bp, Artificial Sequence
  pH3DZ9 - random
ggaagttggagggttcgtattgctacgttgccttagagaggttgtggaagagcggcacatcattgttggg

SEQ ID NO: 52 - DNA, 70 bp, Artificial Sequence
  pH3DZ10 - random
tgaatgaaacctcgggcataaattacggaaacggctttaattttttagtggaaagatccgataacgaggt

SEQ ID NO: 53 - DNA, 68 bp, Artificial Sequence
  pH3DZ11 - random
gaagtgggggtcgggggaagggaggcacgcgtaaaggtaggtgtgagggcgggtgagagttggacaat

SEQ ID NO: 54 - DNA, 70 bp, Artificial Sequence
  pH4DZ1 - random
tgtatgctagctcggggagaaacatctttgcgggataaggccgccgatagagcggaagcgacttggttgt

SEQ ID NO: 55 - DNA, 70 bp, Artificial Sequence
  pH4DZ2 - random
tgaatagggtcctaggcataaattacgaaaacggctttaatcttttagtggaaaggtccgataacgagtg

SEQ ID NO: 56 - DNA, 70 bp, Artificial Sequence
  pH4DZ3 - random
tgggtaaaaggaaaagatggcggagcgagttgatggcgtgattaggaggaggacttaaaggtggtggttg

SEQ ID NO: 57 - DNA, 68 bp, Artificial Sequence
  pH4DZ4 - random
tgtacggtagggagggtgcaaggtgaatcggattagtttacggaagagtgtgattgagtccgatagct

SEQ ID NO: 58 - DNA, 69 bp, Artificial Sequence
  pH4DZ5 - random
cagtagggcaatttggttgggtttaatgtgatacgaagaaccatatttgcggagttctagccggccgat

SEQ ID NO: 59 - DNA, 69 bp, Artificial Sequence
  pH4DZ6 - random
aagatggggctctgacgaggagtttagcggtgatccctgaggacgtttgttgatggatgtggttgggta

SEQ ID NO: 60 - DNA, 70 bp, Artificial Sequence
  pH4DZ7 - random
gaggtgggggtcgggggaagggaggcacgcgtaaaggtaggtgtgagggcgcatgagggaattggacgat

SEQ ID NO: 61 - DNA, 70 bp, Artificial Sequence
  pH4DZ8 - random
gggagttggaggatccgtactgttacgttgtcttagagagggtgtggaagagcggcacattactgttggg

SEQ ID NO: 62 - DNA, 70 bp, Artificial Sequence
  pH5DZ1 - random
tgaatagggtctcgggcataaattacggaaacggttttaattttctagtggaaaggtccgataacgaggt

SEQ ID NO: 63 - DNA, 69 bp, Artificial Sequence
  pH5DZ2 - random
ctgtgggaggctaagagaggttgtggaagagcggtaaactaatatcagtgttatgacagtggtgattgc

SEQ ID NO: 64 - DNA, 69 bp, Artificial Sequence
  pH5DZ3 - random
cggagtagggggggaaggttgggtaaaggaatttttatgctgttagcaggtctaacggcggtgcagggg

SEQ ID NO: 65 - DNA, 68 bp, Artificial Sequence
  pH5DZ4 - random
ggcagaggaggtgggcgatgagtagtagggggggaaggttgggttcagtttagttgcggttggtatac

SEQ ID NO: 66 - DNA, 70 bp, Artificial Sequence
  pH5DZ5 - random
gaagtgggggtcgggggaagggaggcacgtgtaaaggtaggtgtgagggtgtatggaagagttggacaac

SEQ ID NO: 67 - DNA, 70 bp, Artificial Sequence
  pH5DZ6 - random
tcgtagggggggaaggttgggtggaaggagttagtaagacgattgtactagcggtgagggcagggtgatg

SEQ ID NO: 68 - DNA, 70 bp, Artificial Sequence
  pH6DZ1 - random
atcggacaagggaggcactggaggttgaggtagtgagcgttggttaacgccggacaaagggaagcatggt

SEQ ID NO: 69 - DNA, 70 bp, Artificial Sequence
  pH6DZ2 - random
tcgtaaggggggaaggttgggtggagggagtcagtaagacgattgtactagtggtgagggcaggatgatg

SEQ ID NO: 70 - DNA, 69 bp, Artificial Sequence
  pH6DZ3 - random
gagtgggggtcgggggaagggaggcatgcgtaaaggtaggtgcgagggtgcatgaaagggttgggcaac

SEQ ID NO: 71 - DNA, 69 bp, Artificial Sequence
  pH7DZ1- random
gtggaacccatgatgagccgagttggggtgtgtctctcgtatatggcggaagtgggacaatagttgagt

SEQ ID NO: 72 - DNA, 70 bp, Artificial Sequence
  pH7DZ2 - random
gtcggacaagggaggcactggggattgaggtagtgagcgttggttaacgccggacaaaggggagcatggt

SEQ ID NO: 73 - DNA, 70 bp, Artificial Sequence
  pH7DZ3 - random
taactcattatgaggtgacggagtaccggaagagggagagatgaagggatgggcggttgtgctgtgttgg

SEQ ID NO: 74 - DNA, 69 bp, Artificial Sequence
  pH7DZ4 - random
cccatgatgagagtgctactcggaagagggacatgataggggagggattagatggtgtttattgtgtac

(19) United States
(12) Patent Application Publication
(10) Pub. No.: US 2004/0219581 A1
(43) Pub. Date: Nov. 4, 2004

(54) pH dependent signaling DNA enzymes

(76) Inventors: Zhongjie Liu, Hamilton (CA); John D. Brennan, Dundas (CA); Yingfu Li, Dundas (CA)

Correspondence Address: JAGTIANI + GUTTAG, 10363-A DEMOCRACY LANE, FAIRFAX, VA, 22030, US

(21) Appl. No.: 10/793,190

(22) Filed: Mar. 5, 2004

Related U.S. Application Data
(60) Provisional application No. 60/452,501, filed on Mar. 7, 2003.

(51) Int. Cl. C12Q 1/68; C07H 21/04; C12P 19/34; C12N 9/10; C12N 9/22

(52) U.S. Cl. 435/6; 435/91.2; 435/199; 435/320.1; 435/325; 536/23.2; 435/69.1

(57) ABSTRACT

Methods for the selection of deoxyribozymes that are active at selected pH ranges are provided. The method comprises detection of a ribonucleotide cleavage event. The detection of catalysis is coupled to the generation of a fluorescent signal. Novel deoxyribozymes which are capable of performing catalysis at pH3, pH4, pH5, pH6 and pH7 were isolated using the methods of the present invention.

### FIELD OF INVENTION

The present invention is directed to DNA enzymes and methods of obtaining and using those enzymes. In particular, DNA enzymes that require specific metal ions or function at various pH ranges are described.

### BACKGROUND

Deoxyribozymes are a class of catalysts comprising DNA which have great promise as pharmaceutical agents. In addition, deoxyribozymes can be used as molecular tools for therapy, for diagnostic assays and for detection assays. Several studies have shown that single-stranded DNAs with catalytic or binding functions can be isolated from random-sequence DNA pools by in vitro selection. The catalytic capabilities of DNA can be enhanced through the use of metal ions and small-molecule cofactors as well as through modification with new chemical functionalities. DNA has extraordinary chemical stability making it suitable for the development of enzymes for practical applications.

Although this chemical stability suggests that robust catalysts could be developed to operate under physically demanding conditions such as high pH, low pH or extreme high or low temperature, all known deoxyribozymes reported to date function only at or near mild reaction conditions. There have been no previous reports of DNA enzymes which are active under demanding reaction settings. Not all reactions can be carried out at neutral pH and thus, there was a need to engineer efficient DNA catalysts that can function under demanding reaction conditions.

### SUMMARY OF THE INVENTION

The present invention addresses the need for DNA enzymes that can perform catalysis under chemically demanding conditions such as low or high pH. The present invention also provides fluorescence signaling DNA enzymes with a broad range of pH optima to allow biosensing applications to be done in solutions of varying pH.

In one aspect of the present invention, DNA enzymes which are active under stringent conditions were selected using a two-stage selection and evolution strategy that involved an initial series of selection at pH4 followed by further selection and eveolution at pH values ranging from 3.0 to 7.0.

In another aspect, pH sensitive DNA enzymes were modified to generate a fluorescent signal upon activiation. In order to demonstrate the efficacy of the enzymes and methods of the present invention, an experimental system was designed to select a DNA enzyme cable of cleaving an RNA linkage embedded in a DNA sequence. Basically, a single RNA linkage is flanked by a fluorophore-containing nucleotide and a quencher bearing nucleotide. Upon catalysis the fluorophore is separated from the quencher and a fluorescent signal is generated. The sequences of signaling molecules incorporating the enzymatic sequences are shown in SEQ. ID. NOS. 7-38. The enzymatic sequences without the fluorescent signaling tag are described in SEQ. ID. NOS. 43 to 74.

In another aspect of the invention, several DNA enzymes functional at pH3 are provided. These enzymes are generally referred to herein as pH3DZ1, pH3DZ2, pH3DZ3, pH3DZ4, pH3DZ5, pH3DZ6, pH3DZ7, pH3DZ8, pH3DZ9, pH3DZ10, and pH3DZ11. DNA enzymes active at pH3 are listed as SEQ.ID. NOS. 7-17 and 43-53. The signaling DNA enzymes (SEQ.ID. NOS.7-17) comprises a ribonucleotide linkage flanked by a fluorophore-modified nucleotide and a quencher-modified nucleotide and a catalytic sequence capable of cleaving at the ribonucleotide linkage under pH3 reaction conditions.

In another aspect of the invention, a DNA enzyme is provided which is active at pH4. In a preferred embodiment, the DNA enzyme functional at pH4 comprises a sequence selected from the group consisting of SEQ ID NOS. 18-25 and 54-61. The pH4 responsive DNA enzymes are generally referred to herein as pH4DZ1, pH4DZ2, pH4DZ3, pH4DZ4, pH4DZ5, pH4DZ6, pH4DZ7 and pH4DZ8.

In a preferred embodiment, the DNA enzyme active at pH4 is a signaling DNA enzyme molecule comprising a ribonucleotide linkage flanked by a fluorophore-modified nucleotide and a quencher-modified nucleotide and a catalytic sequence capable of cleaving at the ribonucleotide linkage at pH4.

In another aspect of the invention, a DNA enzyme is provided which is active at pH5. The pH5 active DNA enzyme preferably has a sequence selected from the group consisting of SEQ. ID. NOS. 26-31 and 62-67. These enzymes are generally referred to herein as pH5DZ1, pH5DZ2, pH5DZ3, pH5DZ4, pH5DZ5, and pH5DZ6.

The signaling DNA enzyme comprises a ribonucleotide linkage flanked by a fluorophore-modified nucleotide and a quencher-modified nucleotide and a catalytic sequence capable of cleaving at the ribonucleotide linkage at pH5.

In another aspect of the invention, a signaling DNA enzyme is provided which is active at pH6. These DNA enzymes comprise sequences selected from SES.ID.NOS. 32-34 and 68-70. The signaling DNA enzyme comprises a ribonucleotide linkage flanked by a fluorophore-modified nucleotide and a quencher-modified nucleotide and a catalytic sequence capable of cleaving at the ribonucleotide linkage at pH6. pH6 responsive enzymes are referred to herein as pH6DZ1, pH6DZ2 and pH6DZ3.

In another aspect of the invention, a DNA enzyme is provided which is active at pH7. pH7 responsive DNA enzymes comprise sequences selected from SEQ.ID.NOS. 35-38 AND 71-74. A signaling DNA enzyme comprises a ribonucleotide linkage flanked by a fluorophore-modified nucleotide and a quencher-modified nucleotide and a catalytic sequence capable of cleaving at the ribonucleotide linkage at pH7. Specific pH7 sensitive DNA enzymes are referred to herein as pH7DZ1, pH7DZ2, pH7DZ3 and pH7DZ4.

The present invention provides a method for the selection of pH sensitive deoxyribozymes. The method comprises the steps of:

i) providing a population of nucleic acid molecules, each molecule comprising a region of random sequence linked to a region of sequence having a ribonucleotide flanked by a fluorophore-modified nucleotide and a quencher-modified nucleotide;

ii) incubating said population, in the presence of required co-factors, under pre-determined pH conditions;

iii) isolating a sub-population of nucleic acid molecules having catalytic activity;

iv) amplifying said subpopulation;

v) optionally repeating steps ii) to iv) under specific pH conditions; and

vi) isolating a nucleic acid molecule having catalytic activity at a desired pH.

In a preferred embodiment, the nucleic acid subpopulations are subjected to mutagenesis during several rounds of amplification and selection.

In a further preferred embodiment, the random sequence is a DNA sequence.

In another aspect of the invention, a kit for the selection of pH sensitive deoxyribozymes is provided. The kit comprises:

i) a library nucleotide sequence having an insertion site for a random sequence;

ii) an acceptor nucleotide sequence having a ribonucleotide flanked by a fluorophore-modified nucleotide and a quencher-modified nucleotide;

iii) a template DNA sequence; and

iv) a pair of primers suitable for PCR amplification of the library nucleotide sequence and the acceptor nucleotide sequence.

The kit preferably also includes a primer capable of inserting a ribonucleotide. A cocktail of co-factors is optionally included as well as a buffered solution.

The present invention also provides methods for detecting the presence of metal ions using the DNA enzymes described herein. Methods for determining pH using the DNA enzymes of the present invention are also provided. Microarrays, optic fibres and other analytical tools incorporating he DNA enzymes of the present invention are also provided.

### BRIEF DESCRIPTION OF THE DRAWINGS

FIG. 1A demonstrates schematically the general selection method;

FIG. 1B illustrates schematically the selection of DNA enzymes active under various pH conditions;

FIG. 2 illustrates the nucleotide sequences of exemplary DNA enzymes isolated under different conditions;

FIG. 3A shows the sequence of the DNA molecules used for in vitro selection;

FIG. 3B illustrates the nucleotide sequence of five selected deoxyribozymes;

FIG. 4 is a series of phoshorimages and fluoroimages indicating the metal requirements of the selected deoxyribozymes;

FIG. 5 illustrates the pH profiles of selected deoxyribozymes;

FIG. 6 illustrates the real-time signaling capacity of selected deoxyribozymes;

FIG. 7A illustrates the secondary structure of one deoxyribozyme and modifications of that structure;

FIG. 7B illustrates the catalytic activity of the predicted and modified structures; and

FIG. 7C illustrates the fluorescent signaling capability of two trans-acting enzyme systems.

### DETAILED DESCRIPTION

DNA enzymes have many potential applications for the detection of various species. DNA enzymes are particularly suitable in sensitive assays since DNA has exceptional stability and new DNA enzymes with specific properties can be obtained by artificial evolution.

Throughout this application, the terms DNA enzyme, deoxyribozyme, DNAzyme, zymogenic DNA and catalytic DNA are used interchangeably.

Although deoxyribozymes have been reported to catalyze chemical transformations under mild conditions, deoxyribozymes which are functional under chemically demanding conditions, such as highly acidic or highly basic conditions, have not been previously described. The present invention provides the first demonstration that DNA enzymes active at specific pH conditions can be isolated and characterized.

In one aspect, the present invention provides a modified fluorescence signaling selection scheme for the identification of signaling DNA enzymes with different pH optima. The DNA enzymes of the present invention indicate that DNA has the ability to catalyze reactions under extreme pH conditions and DNA enzymes can be created for unique applications that demand acidic pH values.

The present invention provides a combined selection and evolution approach which can be used to fine tune a given property of a pool of DNA catalysts. The DNA catalysts of the present invention are useful in several pratical applications. For example, since certain deoxyribozymes exhibit catalytic activity only in the presence of selected divalent metal ions, the DNA enzymes can be used as sensitive signaling probes to detect specific metal ions at a given pH. The DNA enzymes can also be used as unique pH-reporting probes, either in solution or after immobilization onto a surface. The DNA enzymes of the present invention can also be used as pH dependent signaling probes to follow chemical or enzymatic reactions that alster the pH of a reaction mixture. The signaling DNA enzymes of the present invention can also be engineered into signaling allosteric deoxyribozymes and used as catalytic and real-time reporters in a variety of detection applications over a wide range of pH values. The DNA enzymes of the present invention have significant advantages over prior molecules in that both the catalytic and signaling features are combined in a single molecule. This enables the development of reagentless sensors based on immobilization of the DNAzyme onto a suitable surface such as an optical fibre or microarray.

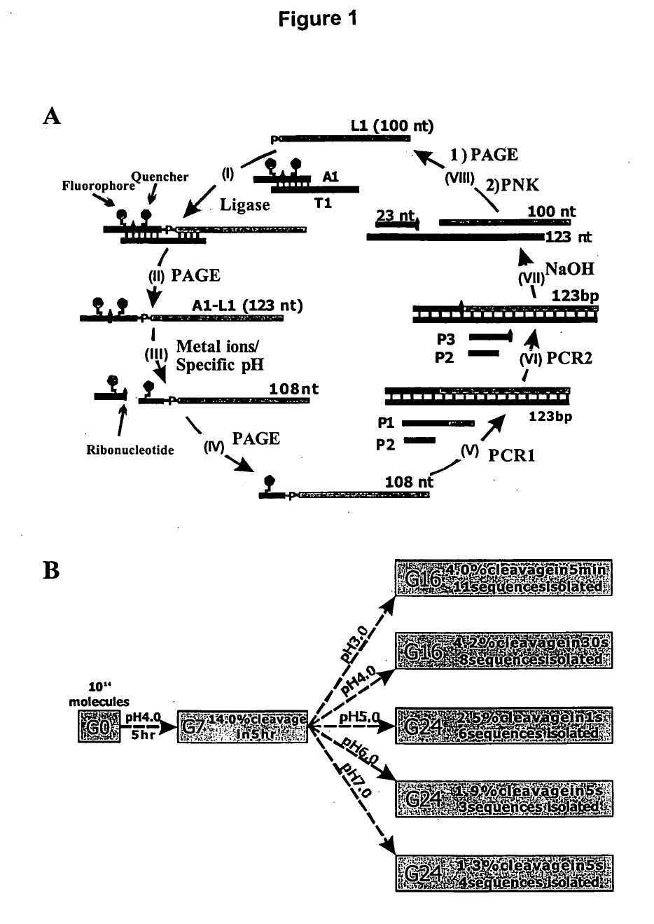

A scheme for the selection of pH specific DNAzymes is shown in FIG. 1A. In the selection scheme shown in FIG. 1, a catalytic event by a DNA enzyme is directly coupled to a fluorescence-signaling event. An RNA-cleaving DNA enzyme, capable of cleaving a single RNA linkage flanked by a fluorophore-containing nucleotide and a quencher-bearing nucleotide, is detected by the generation of a fluorescent signal when the ribonucleotide linkage is cleaved. This type of construct provides a synchronization of the RNA cleavage with fluorescence signal generation (by fluorescence dequenching).

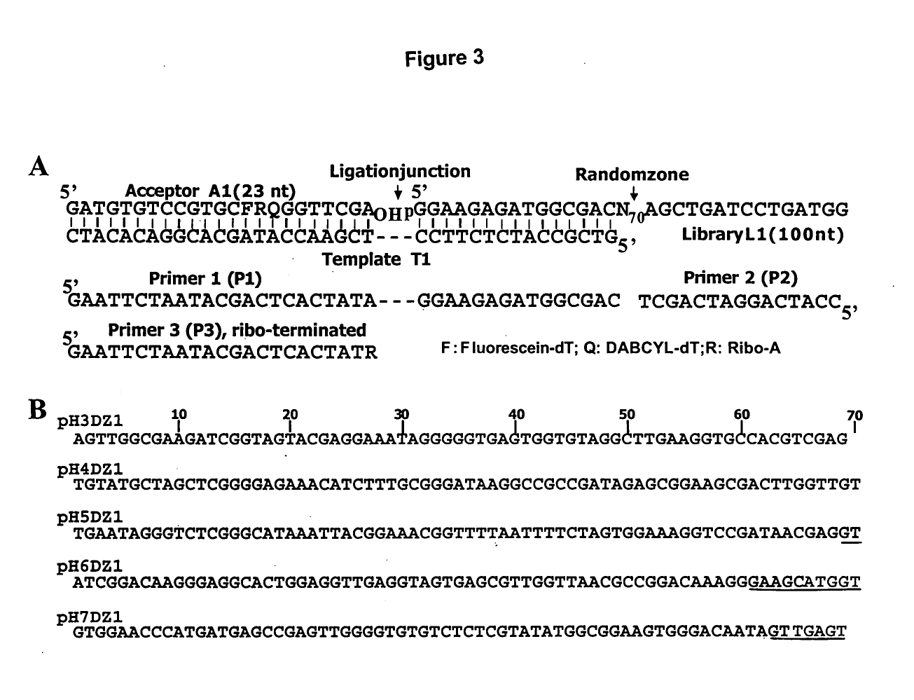

The selection scheme may be more clearly understood by referring to the exemplary scheme outlined in FIGS. 1A and 3A and Example 3. A pool of single stranded DNA molecules comprising a random sequence flanked by a predetermined 5′ sequence and a predetermined 3′ sequence is generated. These DNA molecules are referred to as “library” DNA. One exemplary library DNA molecule has the nucleotide sequence identified in SEQ.ID.NO.2 An oligonucleotide, referred to herein as an “acceptor” oligonucleotide, comprises a fluorophore-modified nucleotide, a quencher modified nucleotide and a ribonucleotide linkage positioned between the fluorophore and the quencher. One such acceptor molecule has the sequence identified in SEQ.ID.NO. 1. Another oligonucleotide, termed “template DNA” is also provided. Template DNA comprises a first sequence which is at least partially complementary to the sequence of the acceptor oligonucleotide and a second sequence which is at least partially complementary to the predetermined 5′ sequence of the library DNA. One such template DNA comprises the sequence of SEQ.ID.NO. 3. Due to the complementarities of the sequences, the template DNA forms a duplex structure with the acceptor oligonucleotide and the library DNA and brings them into proximity. When a ligase is introduced, the library DNA is ligated to the acceptor oligonucleotide to form a ligated molecule. The duplex structure is dissociated and the ligated molecule can be separated from the template DNA by PAGE. The ligated DNA is incubated in the presence of co-factors such as the metal ions, Mn2+, Cd2+, Ni2+ in addition to Mg2+, Na+, and K+.

DNA molecules that are responsive to those ions under the pH conditions employed in the selection will cleave the molecule at the ribonucleotide linkage. This will result in the generation of a fluorescent signal as the fluorophore and quencher become separated. The autocatalytic molecules can then be enriched through a series of polymerase chain reaction amplifications. Since the autocatalytic DNA will have the predetermined 3′ sequence of the library DNA, a primer complementary to that sequence can be used. This primer is termed P1. One such P1 primer has the sequence of SEQ.ID.NO. 4. A second primer, P2, comprises a sequence complementary to the acceptor oligonucleotide and the conserved 5′ sequence of the pool DNA. An exemplary P2 primer has a sequence as defined in SEQ.ID.NO. 5. PCR with these primers will generate DNA molecules having the sequence of the ligated DNA with the exception of the ribonucleotide. The ribonucleotide is then introduced using a third primer, P3, which is ribo-terminated. One example of a P3 primer has the sequence SEQ.ID.NO. 6. After amplification, the DNA is treated with an RNA cleaving moiety, such as NaOH. The cleaved DNA is subjected to PAGE purification and DNA phosphorylation. The 5′ phosphorylated DNA is used to initiate a further round of selection. It is clearly apparent that various library, acceptor, template and primer sequences, other than the specific sequences identified above, can be used in the present invention provided that in combination they have the appropriate complementarities.

In the present method for the selection of pH sensitive enzymes, the pH of the reaction solution for the cleavage step (Step III) is adjusted using various buffers such as MES or HEPES. Typically, although not necessarily, a single stream of selection at a set pH is first carried out followed by separation of the enzymatic population into sub-populations for enrichment under various pH conditions. In the exemplary selection protocol illustrated in FIG. 1B and discussed in greater detail in Example 3, a single stream selection was carried out at pH4. After eight rounds of selection and amplification, the pool was divided into sub-pools for reaction at pH3, pH4, pH5, pH6 or pH7. It is clearly apparent that the initial selection pH level can be varied.

The method of the present invention optionally incorporates in vitro evolution techniques. For example, a hyper-mutagenic PCR protocol can be used to introduce a high rate of mutations in each pH stream. Each stream goes through further rounds of selection. There are preferably at least three further selection rounds, more preferably at least five. The mutagenesis allows the catalytic molecules to acquire mutations so that their structure and function can be adjusted in response to changes in pH. The time for the cleavage step can be progressively reduced to select the most active DNA enzymes. In this manner, catalytic DNA populations from each pH stream can be derived.

An exemplary selection progress chart is shown in FIG. 1B and the selection process is described more fully in Example 3. In this selection, a relatively long reaction time (5 hr) was used in the initial 8 rounds of selection (prior to the pool splitting) with the intention to establish a diverse catalytic DNA pool for the subsequent evolution experiments. The reaction time was first reduced to 10 minutes during the mutagenic rounds of selection and then progressively dropped to as little as 1 sec as long as the relevant catalytic population registered a positive response in RNA cleavage activity. If there was no noticeable activity increase for at least three consecutive rounds at a chosen reaction time, a stream was stopped. For pH3 and pH4 streams, 8 more rounds were conducted after pool splitting while for the pH5 to pH7 streams, 16 more rounds were performed. Five catalytic DNA populations were derived that underwent efficient RNA cleavage at a given pH. The selection progress is summarized in FIG. 1B.

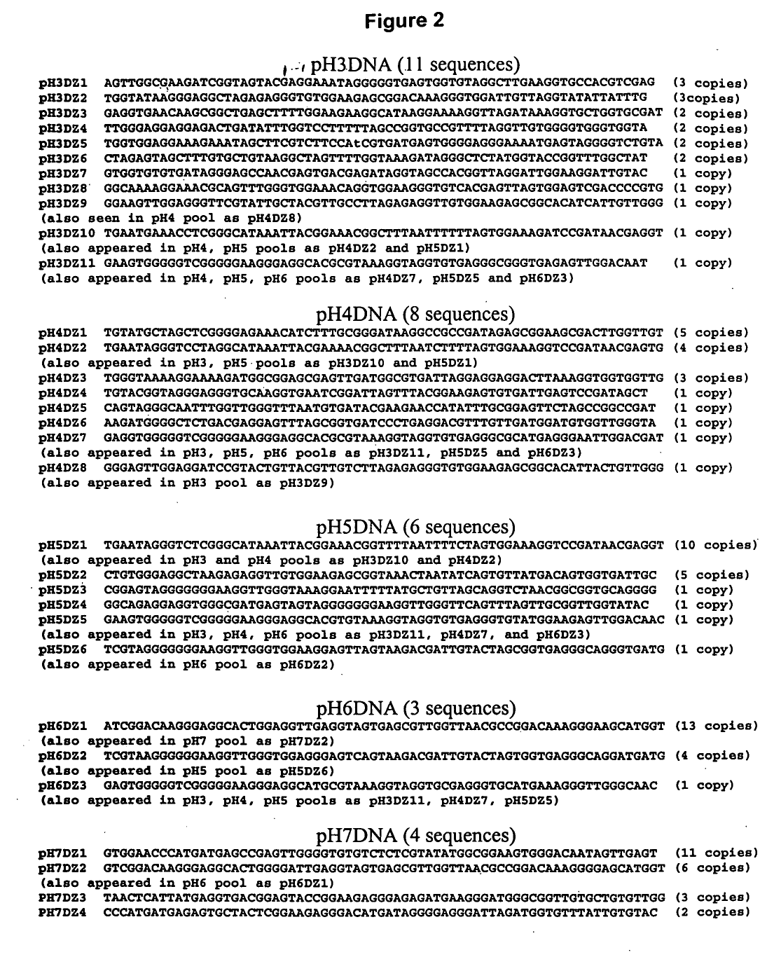

A number of clones from each stream can then be amplified and sequenced using standard protocols well known to those skilled in the art. The sequences of several clones isolated from one such selection process are shown in FIG. 2 and discussed further in Example 4. The sequences of various DNA enzymes identified in the present invention are listed as SEQ.ID.Nos. 7-74. Eleven, eight, six, three and four different sequences were isolated from the pH3 to pH7 pools, respectively, after ˜20 clones were sequenced from each population. Sequences corresponding to SEQ.ID.NOS. 7-17 and 43-53 are active at pH3. Sequences corresponding to SEQ.ID.NOS. 18-25 and 54-61 are active at pH4. Sequences corresponding to SEQ.ID.NOS. 26-31 and 62-67 are active at pH5. Sequences corresponding to SEQ.ID.NOS. 32-34 and 68-70 are active at pH6. Sequences corresponding to SEQ.ID.NOS. 35-38 and 71-74 are active at pH7. The basic DNA enzymes can be converted to signaling DNA enzymes by inserting sequences that include a ribonucleotide linkage flanked by a fluorophore modified nucleotide and a quencher modified nucleotide. The DNA enzymes having SEQ.ID.NOS. 7-38 are signaling DNA enzymes.

Each of the pools contained more than one deoxyribozyme and most DNA catalysts appeared in a single pool and only five deoxyribozymes were observed in two or more populations. These results indicate that diverse deoxyribozymes with wide-ranging pH dependences can be isolated using the methods of the present invention. Twenty-two different deoxyribozymes from ˜100 clones were identified in this way. It is clearly apparent that additional deoxyribozymes could be isolated using the methods of the present invention if more clones were analyzed.

Of the five deoxyribozymes which were detected in 2 or more pools, one appeared in four consecutive pH pools (as pH3DZ11, pH4DZ7, pH5DZ5 and pH6DZ3), another was found in three pH selections (as pH3DZ10, pH4DZ2 and pH5DZ1), and the remaining three were seen in two neighboring pH pools (pH3DZ9 and pH4DZ8, pH5DZ6 and pH6DZ2, pH6DZ1 and pH7DZ2). No single deoxyribozyme was observed in all DNA pools indicating that there was indeed a pH optimum for the various enzymes. Considerable mutations were observed with these DNA catalysts. For example, for pH3DZ11 and its variants in the other 3 DNA pools, base mutations were observed in a total of 13 positions throughout the original random-sequence domain. These results indicate that the selection process of the present invention can be used to isolate novel deoxyribozymes with wide ranging pH dependencies. It is clearly apparent that, if more clones are sequenced, the number of potential deoxyribozymes is increased.

Fluorescence signaling DNA enzymes are provided that have a wide range of pH optima and metal ion specificities. FIG. 3A illustrates the sequences of exemplary DNA molecules that can be used in the selection process. It is clearly apparent that sequences other than the specific sequences shown can be used. In the examples illustrated in FIG. 3, the signaling capacity of the deoxyribozymes of the present invention is imparted by the presence of a ribonucleotide linkage flanked by a fluorophore-modified nucleotide (14th nucleotide) and a quencher modified nucleotide (16th nucleotide). It is clearly apparent, however, that the DNA enzyme sequences identified herein could also be modified with other labels to provide other types of signaling molecules such as radioactive or colorimetric molecules.

Deoxyribozyme sequences isolated according to the method described above can be further characterized. To determine the minimal sequence required for activity, 3′ truncated mutants can be prepared by standard methods, such as chemical synthesis, and then tested for catalytic activity. A prevalent deoxyribozyme from each stream was selected for study. It is clearly apparent that this type of analysis could be applied to any deoxyribozyme selected according to the above-described methodology. Truncated molecules retaining enzymatic activity are encompassed within the scope of the present invention.

FIG. 3B illustrates some examples of truncated molecules that retain activity. The original random sequence domain is shown with the non-essential nucleotides of these exemplary molecules underlined. The truncation experiments are discussed more fully in Example 4. These results indicate that some deoxyribozymes require more nucleotides at the 3′ end to assume a tertiary structure for catalysis.

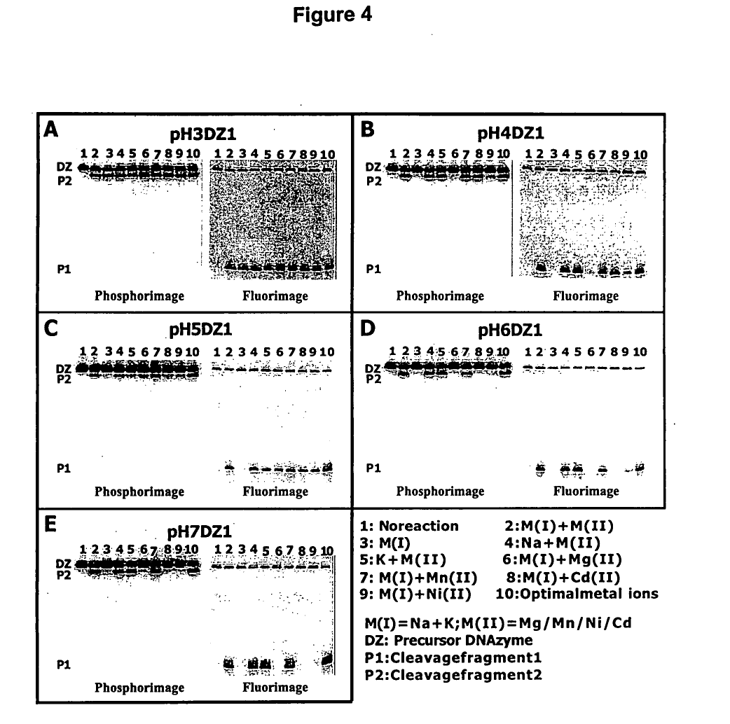

The novel constructs of the present invention make it possible to determine the metal ion specificity of various deoxyribozymes. This can be determined using a variety of methods. In one method, each deoxyribozyme is labeled with 32P in addition to the fluorescein dT, ribo A and DABCYL-dT trio. In a preferred embodiment based on the construct shown in FIG. 3A, 32P is added at the phosphodiester bond linking the 24th and 25th nucleotides. The resultant deoxyribozyme is capable of generating both a radioactive and a fluorescent signal. The deoxyribozyme is then incubated in the presence of various metals. If RNA cleavage occurs, the fluorescein and the 32P labels are separated onto two different fragments. Two products are detectable: a large DNA fragment that is only radioactive and a small DNA fragment that is only fluorescent. FIG. 4 illustrates the results of one such experiment using a deoxyribozyme from each pH stream. The protocol is discussed in greater detail in Example 5. The results of this experiment indicate that the five deoxyribozymes studied exhibited a broad metal ion dependency. All the deoxyribozymes, except pH3DZ1, require divalent metal ion cofactors (lane 1: no reaction; lane 2: full set of divalent metal ions and monovalent metal ions; lane 3: only monovalent metal ions). pH7DZ1 is extremely specific for Mn2+ (lane 7). In contrast, pH5DZ1 is a non-selective metallo enzyme with a slight preference for Mn2+. pH4DZ1 also appears to be non-metal-selective; it has a high catalytic activity with Mn2+ and Cd2+ and a reduced activity with Ni2+ but it is inactive in the presence of only Mg2+. pH6DZ1 appears to require both Mn2+ and Ni2+ for optimal activity and appears incapable of using Mg2+ and Cd2+. The DNA enzymes of the present invention can therefore be used as sensitive signaling probes to detect the presence of certain ions at various pH values.

Additional experiments were performed to establish metal ion concentrations that support the most optimal catalysis for each deoxyribozyme (the optimized conditions are discussed in Example 2). Lane 9 of each gel showed the reaction mixture obtained under the established optimized condition for each deoxyribozyme.

The unique signaling properties of the deoxyribozymes of the present invention make it possible to rapidly identify metal ion requirements.

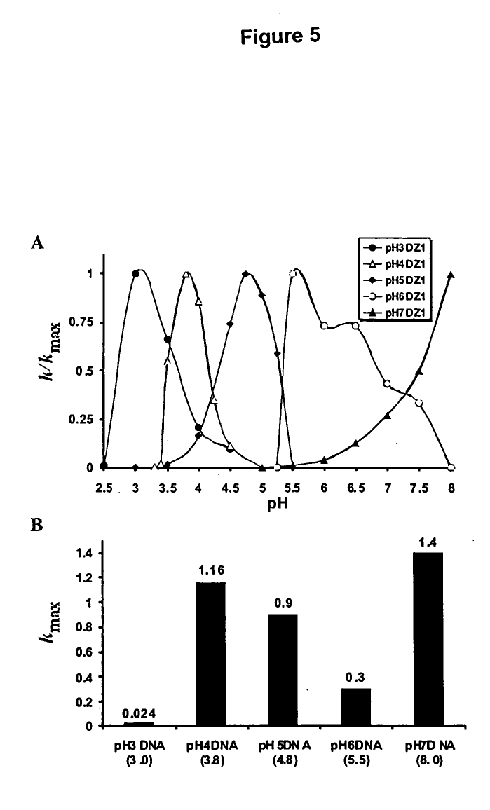

The signaling properties of the unique constructs of the present invention enable one to determine the pH profile of any deoxyribozyme. FIG. 5A illustrates the pH dependence of certain selected DNA enzymes. FIG. 5B illustrates the maximum catalytic rate constant for each of the selected deoxyribozymes. These experiments are discussed further in Example 6. The results indicate that the enzymes selected at pH values of 3 to 6 show corresponding maximum catalytic rate constants at pH values that are at or near the selection pH. The pH3 to 6 systems show relatively narrow pH windows, bracketing 1.5 to 2.75 pH units. In this experiment, the only system that did not show a pH maximum is pH7DZ1, whose catalytic rate rose with increasing pH.

The maximum catalytic rate constants for different deoxyribozymes may vary. In most cases (pH4-7 deoxyribozymes) the enzymes show fairly large rate constants (kobs values range from 0.3 to 1.4 min-1). The pH3 enzymes appear to be less efficient. This may be due to the fact that much of the phosphate backbone and bases would be expected to be protonated at this pH, which might affect the ability of the DNA molecule to fold into catalytically active structures. This speculation draws support from the fact that pH3DZ1 does not require a divalent cation for catalytic activity.

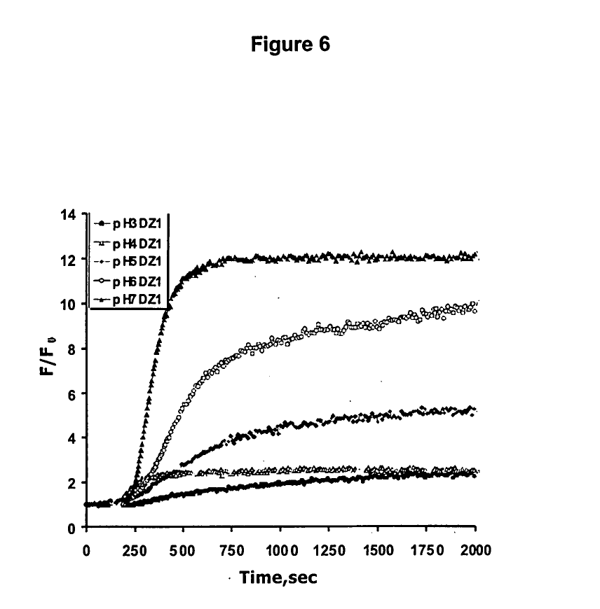

The signaling enzymes of the present invention can also be used to determine the real-time fluorescence signaling capabilities of autocatalytic DNAs under conditions at which optimal catalytic rate constants are observed. Several exemplary pH dependent deoxyribozymes were assessed and the results are shown in FIG. 6 and discussed further in Example 7. Briefly, essential metal ions were added to initiate catalysis at 120 s. In each case, the fluorescence signal rose quite rapidly toward a final plateau value at a rate that mirrors the relative kobs values of the specific enzymes. The net increase in fluorescence intensity is dependent on the pH of the solution utilized for the analysis. In cases where analysis is done at pH7, fluorescein exists predominantly in the dianionic form, and as such has a large emission yield (F=0.93). At pH5, fluorescein exists predominantly as a monoanion, and thus has a yield that is 2.5 fold lower than the dianion (F=0.37). At pH values of 3 and 4 the probe exists predominantly as a non-fluorescent neutral species, which is able to undergo deprotonation in the excited state to elicit monoanion emission. Since quenching by energy transfer to DABCYL must compete with all other forms of quenching (including internal conversion, which is enhanced for the monoanion and neutral forms relative to the dianion), the degree of quenching by the energy transfer mechanism is reduced at lower pH values, leading to a reduced overall enhancement. Even so, the enhancement at lower pH values is reasonable (>2-fold) and is sufficient to provide a useful pH dependent signal. Although specific enzymes have been identified herein, it is clearly apparent that the methods of the present invention can be used to identify other pH dependent DNA enzymes.

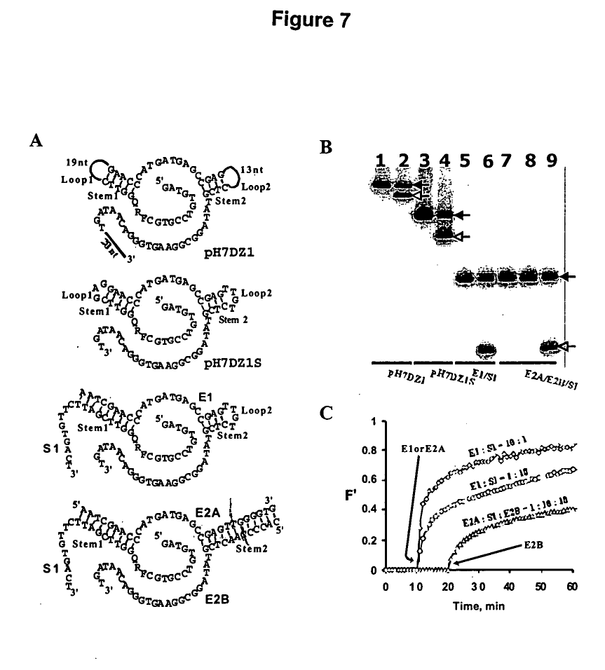

Once the primary sequence is known for a deoxyribozyme, the secondary structures can be predicted for each deoxyribozyme by the M-fold program (data not shown; M-fold program can be accessed at http://bioinfo.math.rpi.edu/˜mfold/dna/form1.cgi). Various synthetic DNA molecules were synthesized to test some of the predicted structures. The identities of selective base pairs were changed in the predicted stems and selective large loops were replaced with 3- or 4-nt loops. The experimental details are discussed in Example 8 below. Although most altered DNA molecules were no longer catalytically active, one of the predicted secondary structures for pH7DZ1, which is shown in FIG. 7A can be modified. In its predicted secondary structure, pHDZ1 has two stem-loop motifs (stem 1/loop 1 and stem 2/loop 2). This proposed structure is supported by the data shown in FIGS. 7B and 7C. A significantly shortened version of pH7DZ1, denoted pH7DZ1S in which 19-nt original loop 1 was replaced by a GAG triloop and 13-nt loop 2 by a TTGT tetraloop along with the deletion of 20 nucleotides from the 3′-end, maintained the full catalytic activity (FIG. 7B, lanes 3 and 4). The existence of stem 1 was confirmed through the use of an engineered trans-acting DNA enzyme denoted E1 that was shown to cleave the matching external substrate S1 (lanes 5 and 6). Similarly, the existence of stem 2 was verified through the use of a bipartite deoxyribozyme assembly, E2A/E2B, that was able to cleave S1 (lanes 7-9). Finally, the two trans-acting systems were examined for fluorescence-signaling capability (FIG. 7C). Each system exhibited the expected signaling behavior: for E1/S1, a rapidly increasing fluorescence signal was observed upon the addition of E1 to a S1-containing solution (diamonds, E1: S1=10:1; circles, E1: S1=1:10); for E2A/E2B/S1, fluorescence signaling can only be achieved when both E2A and E2B were added to the S1-containing solution (triangles, E2A: S1: E2B=1:10:10). pHDZ71 was used as an example for this type of analysis. It is clearly apparent that other pH sensitive DNA enzymes can also be analyze in this way.

DNA enzymes are useful in a variety of practical applications, since DNA has exceptional chemical stability and DNA enzymes are easy to obtain through artificial evolution experiments. Although many deoxyribozymes have been reported to catalyze chemical transformation under or near mild reaction conditions, there have been no previous reports of DNA enzymes that are active under harsh reaction settings. The present invention addresses this need. DNA enzymes which are capable of performing catalysis under chemically demanding conditions such as a high acidity are provided. The methods of the present invention open the door for the development of catalytic DNAs that can catalyze reactions under extreme conditions and the creation of “extremophile” DNA enzymes that are akin to the proteins that are produced by organisms that exist under extreme temperature, pressure, pH or ionic strength conditions. The successful creation of five catalytic DNA populations each are functional at a set pH from a single catalytic pool established at pH 4 indicates that combined in vitro selection and in vitro evolution approach is very powerful in fine-tuning particular properties of DNA catalysts. In the present invention, metal ion specificity was dependent on the selection pH. While divalent metal ions are required by the most deoxyribozymes that were examined, pH3DZ1 does not require divalent metal ions for catalysis.

The signaling DNA enzymes with broad pH optima and metal ion dependences of the present invention have many potential applications. Many of the examined deoxyribozymes exhibit catalytic activity only in the presence of selective divalent metal ions, such as Mn2+, Ni2+ or Cd2+. Thus, these DNA enzymes could be developed into sensitive signaling probes to detect specific divalent metal ions at a given pH. In addition, the DNA enzymes of the present invention are useful as unique pH-reporting probes, either on a surface or in solution. A further application is the use of these signaling probes as pH-dependent fluorogenic reagents to follow chemical or enzymatic reactions that alter the acidity of a reaction mixture. For example, the shifts in pH toward more basic values by urease-catalyzed hydrolysis of urea could be followed with the use of pH7DZ1, leading to a fluorescence enhancement of up to 14 fold. Furthermore, in view of many recent studies showing that ribozymes and deoxyribozymes can be designed into allosteric nucleic acid enzymes and used as effective biosensors for the detection of important biological targets, it is apparent that the signaling DNA enzymes reported herein can be further engineered into various signaling allosteric deoxyribozymes and used as catalytic and real-time reporters in a variety of detection-directed applications. A significant advantage of the signaling DNA enzymes of the present invention is that both the catalytic and signaling components are present in a single molecule. This provides the potential for the development of “reagentless” sensors based on immobilization of the DNAzyme onto a suitable surface such as that of an optical fiber or a microarray. These DNAzymes are also suitable for metal biosensors. In addition to the field of drug screening/biotech, the DNAzymes of the present invention are useful for detection of particular species in environmental and/or waste applications. It is clearly apparent that, in addition to fluorescent signaling, the DNA enzymes of the present invention can be coupled to other agents to provide a different type of readout (e.g. radioactive, colorimetric, density, etc.)

A kit for the isolation of pH sensitive deoxyribozymes according to the methods of the present invention is also contemplated within the scope of the invention. The kit typically comprises the components shown in FIG. 3A. The specific sequences of the DNA molecules used may vary, but will generally include a library nucleotide sequence having an insertion site for a random sequence; an acceptor nucleotide sequence having a ribonucleotide flanked by a fluorophore-modified nucleotide and a quencher-modified nucleotide; a template DNA sequence; and primers suitable for PCR amplification of the library nucleotide sequence and the acceptor nucleotide sequence.

The kit preferably also includes a primer capable of inserting a

ribonucleotide. A cocktail of co-factors is optionally included as well as a buffered solution.

The above disclosure generally describes the present invention. A more complete understanding can be obtained by reference to the following specific Examples. These Examples are described solely for purposes of illustration and are not intended to limit the scope of the invention. Changes in form and substitution of equivalents are contemplated as circumstances may suggest or render expedient. Although specific terms have been employed herein, such terms are intended in a descriptive sense and not for purposes of limitation.

### EXAMPLES

The examples are described for the purposes of illustration and are not intended to limit the scope of the invention.

### Example 1

### Oligonucleotides

Standard and modified oligonucleotides were prepared and purified using the procedures previously described in copending application PCT/CA03/00198. Nucleoside 5¢-triphosphates, [g-32P]ATP and [a-32P]dGTP were purchased from Amersham Pharmacia. Taq DNA polymerase, T4 DNA ligase and T4 polynucleotide kinase (PNK) were purchased from MBI Fermentas. All other chemical reagents were purchased from Sigma and used without further purification.

### Example 2

### Detection of RNA Cleavage

RNA cleavage during in vitro selection and subsequent kinetic analyses was carried out at room temperature (23° C.) in the presence of the following metal ions if not otherwise specified: 400 mM NaCl, 100 mM KCl, 8.5 mM MgCl2, 5 mM MnCl2, 1.25 mM CdCl2, and 0.25 mM NiCl2. The total DNA concentration in each reaction was estimated to be between 0.1 and 0.3 mM. The solution pH was controlled with the following buffering reagents (each used at ˜50 mM): citrate for pH2.5-5.5, MES for pH 5.5-6.5, HEPES for pH 6.5-8.0.

Each full-length, cis-acting DNA catalyst used in PAGE-based kinetic analyses was pieced together by ligation of the substrate A1 and ˜100-nt synthetic deoxyribozyme using DNA template T1 and T4 DNA ligase (all DNA sequences are shown in FIG. 3). Prior to DNA ligation, each deoxyribozyme was phosphorylated with PNK in the presence of g-32P[ATP]. Each ligated DNA catalyst was further purified by 10% denaturing PAGE prior to use. The cleavage reaction was stopped by the addition of EDTA to 30 mM and urea to 8M. The cleavage mixture was analyzed using denaturing 10% PAGE. A phosphorimage (taken on a Storm 820 Phosphorimager, Molecular Dynamics) and a fluorimage (taken on a Typhoon 9200, Molecular Dynamics) were obtained following gel electrophoresis.

DNA molecules used in fluorescence experiments were produced in a similar way except that standard ATP was used to replace g-32P[ATP] in the phosphorylation step. Fluorescence measurements were made on a Cary Eclipse Fluorescence Spectrophotometer (Varian) using a small volume cuvette containing 50 μl of a 100 nM DNA solution. The excitation was set at 490 nm and emission at 520 nm.

The optimal metal ions and pH for each catalytic DNA were determined. These are 500 mM NaCl and pH 3.0 for pH3DZ1; 400 mM NaCl, 10 mM Cd2+ and pH 3.8 for pH4DZ1; 250 mM NaCl, 25 mM Mn2+ and pH4.8 for pH5DZ1; 800 mM NaCl, 8 mM Mn2+, 2 mM Ni2+ and pH 5.5 for pH6DZ1; 100 mM NaCl, 14 mM Mn2+ and pH 8.0 for pH7DZ1.

### Example 3

### In Vitro Selection

A generalized selection scheme for catalytic DNA molecules is shown in FIG. 1A. The specific sequences of the DNA molecules used are shown in FIG. 3A. The selection protocol is generally based on the protocol described in co-pending application no. PCT/CA03/00198. In the present invention, 275 pmol of DNAs each containing a 70-nt random-domain was used as the initial pool. The RNA cleavage reaction in the first 8 rounds (G0-G7) was allowed to proceed for 5 hr at pH4.0. The G8 DNA was then split into 5 pools and 5 streams of selection were carried out at pH 3, 4, 5, 6 and 7, respectively (denoted pH3 stream, pH4 stream and so on). A hyper-mutagenic PCR protocol was used to introduce a high rate of mutations (up to 10% per base per generation) in each stream for five consecutive rounds following the pool splitting (i.e., G8-G13). The cleavage time was progressively reduced from the initial 5 hr (G7) to 30 min (G8-G10, all streams), to 5 min (G11-G16, pH3 stream; G11-G13, all other streams), to 30 s (G14-G16, pH4-7 streams), to 5 s (G17-G24, pH6-7 streams; G17-G21, pH5 stream), and finally to 1 s (G22-G24, pH5 stream). Each selection stream was discontinued if no significant increase of cleavage activity was observed over at least 3 consecutive rounds at a given cleavage time. DNA sequences from each terminal round (G16 for pH3-4 streams; G24 for pH5-7 streams) were amplified by PCR, cloned and sequenced.

### Example 4

### Sequence Truncation

Full-length DNA catalysts and their shortened versions (with one or several nucleotides truncated from the 3′-end of each deoxyribozyme each time) were compared for RNA cleavage activity under the original selection conditions FIG. 3B shows the sequences of five selected deoxyribozymes. Only the original random-sequence domain (numbered from 1 to 70) of each catalytic DNA is shown. Each catalytic DNA also contains GATGT GTCC GTGCF RQGGT TCGAG GAAGA GATGG CGAC (F: fluorescein-dT; R: ribo-A; Q: DABCYL-dT) at the 5′-end and AGCTG ATCCT GATGG at the 3′-end. The underlined nucleotides in pH5 DZ1, pH6DNA1 and pH7DZ1 can be truncated without causing a significant reduction in catalytic activity. pH4DZ1 requires the following additional sequence at the 3′-end for catalytic activity: AGCTGA.

The 15 fixed nucleotides at the 3′-end of pH3DZ1 can be removed with affecting the catalytic DNA's activity.

### Example 5

### Metal Requirements

Each catalyst was studied for metal ion requirements in a 30-min cleavage reaction. FIG. 4 demonstrates the metal-ion requirements of five selected deoxyribozymes. The ˜123-nt DNA catalysts contained 32P-phosphodiester bond linking the 23rd nt and 24th nt. Each catalyst was tested for RNA cleavage under various salt conditions. Reaction products were analyzed on 10% denaturing PAGE, which was both scanned for radioactivity (left image) and fluorescence (right image). DZ stands for the full-length DNA, P2 and P1 for the 5′ and 3′ cleavage products, respectively.

The metal ions and their concentrations in connection with the data shown in FIG. 4 were as follows: no metal ions (lane 1); 400 mM Na+, 100 mM K+, 8.5 mM Mg2+, 5 mM Mn2+, 1.25 mM Cd2+ and 0.25 mM Ni2+ (lane 2); 400 mM Na+ and 100 mM K+ (lane 3); 500 mM Na+, 8.5 mM Mg2+, 5 mM Mn2+, 1.25 mM Cd2+ and 0.25 mM Ni2+ (lane 4); 500 mM K+, 8.5 mM Mg2+, 5 mM Mn2+, 1.25 mM Cd2+ and 0.25 mM Ni2+ (lane 5); 400 mM Na+, 100 mM K+, 15 mM Mg2+ (lane 6); 400 mM Na+, 100 mM K+, 10 mM Mg2+, 5 mM Mn2+, (lane 7); 400 mM Na+, 100 mM K+, 14.75 mM Mg2+ and 0.25 mM Cd2+ (lane 8); 400 mM Na+, 100 mM K+, 13.75 mM Mg2+ and 1.25 mM Cd2+ (lane 9); the optimal metal ions and pH as determined in Example 2 above (lane 10).

### Example 6

### pH Profiles

Each catalyst was allowed to undergo the RNA cleavage reaction under the optimal metal ion conditions under several different pH settings. Aliquots of each reaction mixture were collected at various time points within 15% cleavage completion and analyzed by 10% denaturing PAGE. The rate constant was determined by plotting the natural logarithm of the fraction of DNA that remained unreacted vs. the reaction time. Experiments were duplicated (with less than 20% variation) and the average values are plotted in FIG. 5. FIG. 5A shows the normalized catalytic rates in response to pH changes. The catalytic rate constants were determined for each deoxyribozyme at several pH values. The normalized catalytic rates were calculated as follows: k/kmax, where k is the rate constant at a given pH and kmax is the largest rate in each data series. FIG. 5B illustrates the kmax for each deoxyribozyme. The number on each data bar is the kmax (min-1) for the deoxyribozyme. The number in parenthesis under the name of each deoxyribozyme indicates the pH where the kmax, was observed.

### Example 7

### Real-time Signaling

Each catalyst was first incubated in the absence of metal cofactors for 120 seconds (s), followed by the addition of metal ions and a further incubation for 2000 s. The fluorescence intensity was recorded every 2 s. A control sample was also examined at the same time in which A1 was used to replace the deoxyribozyme. Fluorescence enhancement was calculated as F/F0, where F is the fluorescence intensity of the deoxyribozyme solution and F0 is the intensity of the control sample taken at the same time. Optimal metal ions and optimal solution pH were used to obtain the data shown in FIG. 6.

### Example 8

### Proposed Secondary Structure of pH7DZ1

The secondary structure of several pH dependent deoxyribozymes was predicted using the M-fold program and several modifications were introduced to confirm the structure FIG. 7 illustrates modifications to pHDZ1. Referring to FIG. 7A, pH7DZ1 is the full-length cis-acting catalyst. pH7DZ1S (SEQ.ID.NO.39) is a shortened cis-acting deoxyribozyme where the original loops 1 and 2 were replaced with two small loops. E1/S1 is a trans-acting system in which E1 binds S1 through the formation of 8-bp duplex (stem 1). E2A/E2B/S1 is another trans-acting system in which E2B binds E2A through 8-bp stem 2 and E2A in turn binds S1 through 8-bp stem 1. The sequences for E1, E2A and E2B correspond to SEQ.ID.NOS. 40, 41 and 42, respectively. FIG. 7B illustrates the results of cleavage reactions. Lanes 1 and 2 were for pH7DZ1 cis-acting system: pH7DZ1 (0.1 mM) was treated in the reaction buffer without (lane 1) and with Mn(H) (lane 2). Lanes 3 and 4 were for pH7DZ1S cis-acting system: pH7DZ1S (0.1 mM) was treated in the reaction buffer without (lane 3) and with Mn(II) (lane 4). Lanes 5 and 6 were for E1/S1 trans-acting system: S1 (0.01 mM) was incubated in the Mn(II)-containing buffer in the absence of E1 (lane 5) and in the presence of 1 mM of E1 (lane 6). Lanes 7-9 were for E2A/E2B/S1 trans-acting system: S1 (0.01 mM) was incubated in the Mn(II)-containing buffer in the absence of E2A and E2B (lane 7) and in the presence of 1 mM of E2A (lane 8) and in the presence of 1 mM of E2A and 2 mM of E2B (lane 9). FIG. 7C illustrates the real-time signaling capability of E1/S1 and E2A/E2B/S1 systems. For E1/S1 (circles), the substrate S1 (1 mM) was incubated at room temperature in the absence of E1 for 10 min, followed by the addition of E1 to 0.01 mM and a further incubation for 3000 more minutes (only the first 60 minutes are shown); a similar experiment was conducted with S1 at 1 mM and E1 at 0.1 mM (triangles). For E2A/E2B/S1 (triangles), S1 (1 mM) was incubated at room temperature in the absence of both E2A and E2B for 10 min, followed by the addition of E2A to 0.01 mM and a further incubation 10 more minutes, and followed by the addition of E2B to 1 mM and an extended incubation 3000 more minutes (again only the first 60 minutes are shown). The fluorescence intensity was recorded automatically every minute. The fluorescence intensities were normalized using the following equation: F′=(F-F0)/(F3000-F0), where F3000 and F0 are the fluorescence readings taken at the beginning and end of each reaction and F is the reading at any given time. The reaction solution contained 50 mM Tris (pH 8.0, at 23° C.), 400 mM NaCl, 100 mM KCl, 15 mM Mn2+.

1. A DNA enzyme which is functional at a pH<7.

2. A DNA enzyme having a nucleotide sequence selected from the group consisting of SEQ. ID NO. 7, SEQ. ID NO. 8, SEQ. ID NO.9, SEQ. ID NO.10, SEQ. ID NO.11, SEQ. ID NO.12, SEQ. ID NO.13, SEQ. ID NO.14, SEQ. ID NO.15, SEQ. ID NO.16, SEQ. ID NO.17, SEQ. ID NO.18, SEQ. ID NO.19, SEQ. ID NO.20, SEQ. ID NO.21, SEQ. ID NO.22, SEQ. ID NO.23, SEQ. ID NO.24, SEQ. ID NO.25, SEQ. ID NO.26, SEQ. ID NO.27, SEQ. ID NO.28, SEQ. ID NO.29, SEQ. ID NO.30, SEQ. ID NO.31, SEQ. ID NO.32, SEQ. ID NO.33, SEQ. ID NO.34, SEQ. ID NO.35, SEQ. ID NO.36, SEQ. ID NO.37, SEQ. ID NO.38, SEQ. ID NO.39, SEQ. ID NO.40, SEQ. ID NO.41, SEQ. ID NO.42, SEQ. ID NO.43, SEQ. ID NO.44, SEQ. ID NO.45, SEQ. ID NO.46, SEQ. ID NO.47, SEQ. ID NO.48, SEQ. ID NO.49, SEQ. ID NO.50, SEQ. ID NO.51, SEQ. ID NO.52, SEQ. ID NO.53, SEQ. ID NO.54, SEQ. ID NO.55, SEQ. ID NO.56, SEQ. ID NO.57, SEQ. ID NO.58, SEQ. ID NO.59, SEQ. ID NO.60, SEQ. ID NO.61, SEQ. ID NO.62, SEQ. ID NO.63, SEQ. ID NO.64, SEQ. ID NO.65, SEQ. ID NO.66, SEQ. ID NO.67, SEQ. ID NO.68, SEQ. ID NO.69, SEQ. ID NO.70, SEQ. ID NO.71, SEQ. ID NO.72, SEQ. ID NO.73 and SEQ. ID NO. 74.

3. A DNA enzyme according to claim 2 wherein said DNA enzymes is a signaling enzyme and has a nucleotide sequence selected from the group consisting of SEQ. ID NO. 7, SEQ. ID NO. 8, SEQ. ID NO.9, SEQ. ID NO.10, SEQ. ID NO.11, SEQ. ID NO.12, SEQ. ID NO.13, SEQ. ID NO.14, SEQ. ID NO.15, SEQ. ID NO.16, SEQ. ID NO.17, SEQ. ID NO.18, SEQ. ID NO.19, SEQ. ID NO.20, SEQ. ID NO.21, SEQ. ID NO.22, SEQ. ID NO.23, SEQ. ID NO.24, SEQ. ID NO.25, SEQ. ID NO.26, SEQ. ID NO.27, SEQ. ID NO.28, SEQ. ID NO.29, SEQ. ID NO.30, SEQ. ID NO.31, SEQ. ID NO.32, SEQ. ID NO.33, SEQ. ID NO.34, SEQ. ID NO.35, SEQ. ID NO.36, SEQ. ID NO.37 and SEQ. ID NO.38.

4. A DNA enzyme according to claim 2 wherein said enzyme is active at pH3 and comprises a sequence selected from the group consisting of SEQ. ID NO. 7, SEQ. ID NO. 8, SEQ. ID NO.9, SEQ. ID NO.10, SEQ. ID NO. 11, SEQ. ID NO.12, SEQ. ID NO.13, SEQ. ID NO.14, SEQ. ID NO.15, SEQ. ID NO.16, SEQ. ID NO.17, SEQ. ID NO.43, SEQ. ID NO.44, SEQ. ID NO.45, SEQ. ID NO.46, SEQ. ID NO.47, SEQ. ID NO.48, SEQ. ID NO.49, SEQ. ID NO.50, SEQ. ID NO.51, SEQ. ID NO.52, and SEQ. ID NO.53

5. A DNA enzyme according to claim 2 wherein said enzyme is active at pH4 and has a nucleotide sequence selected from the group consisting of SEQ. ID NO.18, SEQ. ID NO.19, SEQ. ID NO.20, SEQ. ID NO.21, SEQ. ID NO.22, SEQ. ID NO.23, SEQ. ID NO.24, SEQ. ID NO.25, SEQ. ID NO.54, SEQ. ID NO.55, SEQ. ID NO.56, SEQ. ID NO.57, SEQ. ID NO.58, SEQ. ID NO.59, SEQ. ID NO.60 and SEQ. ID NO.61.

6. A DNA enzyme according to claim 2 wherein said enzyme is active at pH5 and has a sequence selected from the group consisting of SEQ. ID NO. 26, SEQ. ID NO. 27, SEQ. ID NO. 28, SEQ. ID NO. 29, SEQ. ID NO. 30, SEQ. ID NO. 31, SEQ. ID NO. 62, SEQ. ID NO. 63, SEQ. ID NO. 64, SEQ. ID NO. 65, SEQ. ID NO. 66 and SEQ. ID NO. 67.

7. A DNA enzyme according to claim 2 wherein said enzyme is active at pH6 and has a nucleotide sequence selected from the group consisting of SEQ. ID NO. 32, SEQ. ID NO. 33, SEQ. ID NO. 34, SEQ. ID NO. 68, SEQ. ID NO. 69 and SEQ. ID NO. 70.

8. A DNA enzyme according to claim 2 wherein said enzyme is active at pH7 and has a sequence selected from the group consisting of SEQ. ID NO. 35, SEQ. ID NO. 36, SEQ. ID NO. 37, SEQ. ID NO. 38, SEQ. ID NO. 71, SEQ. ID NO.72, SEQ. ID NO. 73, and SEQ. ID NO. 74.

9. A method for the selection of DNA enzymes active at a selected pH, said method comprising the steps of:
a. obtaining a pool of nucleic acid molecules having an insert of random nucleotides and at least one ribonucleotide linkage;
b. incubating said pool at predetermined pH; and
c. selecting DNA molecules that are cleaved at the ribonucleotide linkage at that pH.

10. The method of claim 9, further comprising the step of amplifying the selected DNA molecules and repeating steps b) and c).

11. The method of claim 10 further comprising the step of sequencing the amplified DNA.

12. The method of claim 10 further comprising mutagenesis during the amplification step.

13. The method of claim 9 wherein the cleaved DNA molecules are separated based on size.

14. The method of claim 9 wherein the DNA pool is immobilized through duplex formation with a complementary sequence and released upon cleavage at the ribonucleotide linkage.

15. A method for the selection of signaling, pH sensitive deoxyribozymes, said method comprising the steps of:
a. providing a population of nucleic acid molecules, each molecule comprising a region of random sequence linked to a region of sequence having a ribonucleotide flanked by a fluorophore modified nucleotide and a quencher nucleotide;
b. incubating said population, in the presence of required co-factors, under predetermined pH conditions;
c. isolating a sub-population of nucleic acid molecules having catalytic activity based upon generation of a fluorescent signal upon cleavage at the ribonucleotide linkage;
d. amplifying said population;
e. optionally repeating steps (b) to (d) under specific pH conditions; and
f. isolating a nucleic acid molecule having catalytic activity at a desired pH.

16. A kit for the selection of pH sensitive deoxyribozymes comprising:
a. a library nucleotide sequence having an insertion site for a random sequence;
b. an acceptor nucleotide sequence having a ribonucleotide flanked by a fluorophore modified nucleotide and a quencher modified nucleotide;
c. a template DNA sequence; and
d. a pair of primers suitable for PCR amplification of the library nucleotide sequence and the acceptor nucleotide sequence.

17. The kit of claim 16 further comprising a cocktail of co-factors and a buffered solution.

18. A method of detecting specific divalent metal ions in a sample comprising incubating said sample in the presence of a DNA enzyme as defined in any one of claims 2 to 8 at a specific pH value.

19. A method of determining the pH of a sample comprising incubating said sample in the presence of a pH reporting probe comprising a DNA enzyme as defined in any one of claims 2 to 8.

20. A method of detecting a biological target comprising incubating said target in the presence of a signaling allosteric dioxyribozyme comprising a DNA enzyme as defined in any one of claims 2 to 8.
